(19) United States
(12) Patent Application Publication
(10) Pub. No.: US 2021/0369598 A1
(43) Pub. Date: Dec. 2, 2021

(54) COMPOSITIONS AND METHODS RELATING TO AMNION

(71) Applicant: BIOCEL LIMITED, Cottingham (GB)

(72) Inventors: Maneesh SINGH, Cottingham (GB); Patrick WARNKE, Cottingham (GB); Francis MARTIN, Cottingham (GB)

(21) Appl. No.: 16/963,387

(22) PCT Filed: Jan. 21, 2019
(86) PCT No.: PCT/GB2019/050157

§ 371 Date: Jul. 20, 2020

(30) Foreign Application Priority Data
Jan. 19, 2018 (GB) 1800909.2

(51) Int. Cl. A61K 8/98; A61K 8/04; A61Q 19/08; A61L 27/52; A61L 27/36; A61L 27/54; A61L 15/44; A61L 15/40

(52) U.S. Cl. CPC A61K 8/982; A61K 8/042; A61Q 19/08; A61L 27/52; A61L 2300/414; A61L 27/54; A61L 15/44; A61L 15/40; A61L 27/3604

(57) ABSTRACT

This invention relates to a composition comprising amnion homogenate in admixture with a biologically acceptable excipient such as a hydrogel, which may be useful as a therapeutic or cosmetic composition and in particular as a wound dressing, a scar dressing, a topical hydrogel or a topical ointment. This invention also relates to devices such as wound dressings and cosmetic patches comprising the composition and to methods of making and using the composition or device.

### FIELD OF THE INVENTION

This invention relates to compositions and devices comprising amnion, and to methods for their manufacture and use.

### BACKGROUND TO THE INVENTION

Approximately 234 million major surgical procedures are performed every year worldwide with an estimated 295 to 11,110 procedures performed per 100,000 of the population (Weiser T G, Regenbogen S E, Thompson K D, Haynes A B, Lipsitz S R, Berry W R, Gawande A A. An estimation of the global volume of surgery: a modelling strategy based on available data. Lancet 2008 Jul. 12; 372(9633):139-144). In an era of an ageing and rising population, it remains essential to improve surgical outcomes, to reduce not only morbidity but also the economic costs associated with the increasing prevalence of surgery.

In addition, obesity rates have risen dramatically over the last four decades (Ogden C L, YanovskiSZ, Carroll M D, Flegal K M. The epidemiology of obesity. Gastroenterology 2007; 132(6):2087-102 and Flegal K M, Carroll M D, Kit B K, Ogden C L. Prevalence of Obesity and trends in the distribution of body mass index Among US adults 1999-2010. Jama. 2012; 307(5):491-7). This increase is associated with an increase in metabolic and chronic conditions such as diabetes and cancer (Willett W, Dietz W, Colditz G. Guidelines for healthy weight. NEJM. 1999; 341:427-34 and Yu Y. The changing body mass-mortality Association in the United States: Evidence of sex-specific cohort Trends from three national health and nutrition examination Surveys. Biodemography and Social Biology. 2016; 62(2):143-63). Diabetic foot ulcers are a complication of prolonged diabetes and have an approximate prevalence of 6.4% in Type 2 diabetes and 5.5% in Type 1 diabetes (Zhang P, Lu J, Jing Y, Tang S, Zhu D, Bi Y. Global epidemiology of diabetic foot ulceration: a systematic review and meta-analysis. Ann Med. 2016 Nov. 3:1-11). Traditionally these ulcers are difficult to treat, although novel treatments using placental-derived mesenchymal stem cells demonstrate accelerated healing (Wang, H, Chen L, Liu Y, Luo B, Xie N, Tan T, Song L, Erli P, Luo M. Implantation of placental-derived mesenchymal stem cells accelerates murine dermal wound closure through immunomodulation. Am J Transl Res. 2016 Nov. 15; 8(11):4912-4921).

Keloid scars are benign hyperproliferative growths formed of dense fibrous tissue that extends beyond the boundaries of the original wound. Keloid scars are cosmetically displeasing and may lead to pain and restriction of movement. Keloid scars usually present in the first 3 months post-injury, although human keloids may occur up to 1 year post-injury (Brissett A E, Sherris D A. Scar contractures, hypertrophic scars and keloids. Facial Plast Surg 2001: 17:263-272). Keloid scars may be found in all ethnic groups but are seen more commonly in those of African, Asian, Hispanic or Mediterranean descent. Those with darker skin are 15 times more likely to have keloid compared with lighter-skinned individuals (Brissett A E, Sherris D A. Scar contractures, hypertrophic scars and keloids. Facial Plast Surg 2001: 17:263-272), with higher frequencies in puberty and pregnancy (Akoz T, Gideroglu K, Akan M. Combination of different techniques for the treatment of earlobe keloids. Aesthetic Plast Surg 2002; 26:184-88). Increased inflammation is a prerequisite for scarring and disruption in the inflammatory process may lead to increased scarring and thus keloid formation (Martin P, Leibovich S J. Inflammatory cells during wound repair: the good, the bad and the ugly. Trends in Cell Biology 2005; 15:599-607). Modulation of inflammation at this stage may therefore be critical to reducing the incidence of keloid scarring.

A variety of methods have been utilised for reducing keloid scarring, all of which remain unsatisfactory with high recurrence rates and include surgical excision (Poochareon V N, Berman B. New therapies for the management of keloids. J Craniofac Surg 2003; 14:654-57), corticosteroids (Niessen F B, Spauwen P H, Schalkwijk J, Kon M. On the nature of hypertrophic scars and keloids: a review. Plast Reconstr Surg 1999; 104:1435-1458), imiquimod (Jacob S E, Berman B, Nassiri M, Vincek V. Topical application of imiquimod 5% cream to keloids alters expression genes associated with apoptosis. Br J Dermatol 2003; 149:62-65, and Malhotra A K, Gupta S, Khaiatan B K, Sharma V K. Imiquimod 5% cream for the prevention of recurrence after excision of presternal keloids. Dermatology 2007; 215:63-65), interferon-α-2b (Berman B, Duncan M R. Short term keloid treatment in vivo with human interferon α-2b in a selective and persistent normalization of keloid fibroblast collagen, glycosaminoglycan and collagenase production in vitro. J Am Acad Dermatol 1989; 21:694-702), 5 fluorouracil (Gupta S, Kalra A. Efficacy and safety of intralesional 5-fluorouracil in the treatment of keloids. Dermatology 2002; 30:54-56), mitomycin C (Sanders K W, Gage-White L, Strucker F J. Topical mitomycin C in the prevention keloid scar recurrence. Arch Facial Plast Surg 2005; 7:172-175), retinoids (Janssen de Limpens A M. The local treatment of hypertrophic scars with topical retinoic acid. Br J Dermatol 1980; 103:319-323), radiotherapy (Ogawa R, Miyashita T, Hyakusoku H, Akaishi S, Kuribayashi S, Tateno A. Postoperative radiation protocol for keloids and hypertrophic scars: statistical analysis of 370 sites followed for over 18 months. Ann Plast Surg 2007; 59:688-691), laser therapy (Apfelberg D B, Maser M R, Lash H, White D, Weston J. Preliminary results of argon and carbon dioxide laser treatment for keloid scars. Lasers Surg Med 1984; 4:283-290), cryotherapy (Har-Shai Y, Amar M, Sabo E. Intralesional cryotherapy for enhancing the involution of hypertrophic scars and keloids. Plast Reconstr Surg 2003; 111:1841-1852) and occlusive dressings such as silicone therapy (Borgognoni L. Biological effects of silicone gel sheeting. Wound Repair Regen 2002; 10:118-121).

The amnion is a membrane that forms the innermost layer of the amniotic sac that surrounds a developing foetus in most mammals. The amnion is connected to an outer layer of the amniotic sac, the chorion, and can be separated from this outer layer to provide a two-layered membrane with a single layer of epithelial amniocytes. The human amnion is known to be immunologically immature, to reduce inflammation and to alleviate pain, thus providing a natural biological barrier reducing infection and promoting cell proliferation (Zelen C, Serena T E, Denoziere G, Fetterolf D E. A prospective randomized comparative parallel study of amniotic membrane wound graft in the management of diabetic foot ulcers. Int Wound J 2013 DOI: 10.1111/iwj.12097, Ennis W, Sui A, Papineau E, Plummer M, Altman I, Meneses P. Clinical experience with a novel regenerative template for hard to heal wounds. SAWC Annual Spring Meeting in Atlanta, Ga. April 2012, Forbes J, Fetterolf D. Dehydrated amniotic membrane allografts for the treatment of chronic wounds: a case study. J Wound Care 2012; 21:290-6 and Sheikh E S, Sheikh E S, Fetterolf D E. Use of dehydrated amniotic membrane allografts to promote healing in patients with refractory non-healing wounds. Int Wound J 2013. DOI 10.1111/iwj.12035). Although the exact mechanisms of action remain to be fully elucidated, human amnion is known to contain many beneficial molecular constituents such as growth factors and chemokines including epidermal growth factor (EGF), basic fibroblast growth factor (bFGF), keratinocyte growth factor (KGF), transforming growth factors α and β (TGF-α and TGF-β), nerve growth factor (NGF) and hepatocyte growth factor (HGF), all of which are known to promote wound healing and tissue regeneration (Lopez-Valladares M J, Rodriguez-Ares M T, Tourino R, Gude F, Teresa Silva M, Couceiro J. Donor age and gestational age influence on growth factor levels in human amniotic membrane. Acta Opthlmol 2010; 88:211-6 and Russo A, Bonci P, Bonci P. The effects of different preservation processes on the total protein and growth factor content in a new biological product developed from human amniotic membrane. Cell Tissue Bank 2012; 13:353-61).

Human amnion has been shown to be effective in many clinical conditions, including topical application to burns and corneal abrasions (John T. Human amniotic membrane transplantation: past, present and future. Opthalmol Clin North Am 2003; 16:43-65, Cornwell K G, Landsman A, James K S. Extracellular matrix biomaterials for soft tissue repair. Clin Podiatr Med Surg 2009; 26:507-23, Gruss J S, Jirsch D W. Human amniotic membrane: a versatile wound dressing. Can Med Assoc J. 1978; 118:1237-46 and Sawheny C P. Amniotic membrane as a biological dressing in the management of burns. Burns 1989; 15:339-42). However, harnessing the qualities of amnion as an effective and off-the-shelf product poses many problems including infection risk and maintaining its biological action.

Purion® is a processed dehydrated human amnion/chorion tissue allograft produced by gentle cleansing and dehydration, which has been shown to contain the biologically active factors above as well as IL-4, 6, 8 and 10, and TIMP-1, 2 and 4 (Koob T J, Rennert R, Zabek N, Massee M, Lim J J, Temenoff J S, Li W W, Gurtner G. Biological properties of dehydrated human amnion/chorion composite graft: implications for chronic wound healing. Int Wound J. DOI 10.1111/iwj.12140). However, this product must be rehydrated prior to application to a wound, does not provide a consistent effect, especially over uneven wound surfaces, and can only be applied for a relatively short time of approximately 24 hours.

There is therefore a need for a composition that is useful as a wound dressing that supports rapid wound healing and tissue regeneration, provides an effective treatment for slow healing wounds, such as diabetic ulcers, and scars, such as keloid scars, provides an effective means of reducing the appearance of scars, improving the appearance of skin and treating skin disorders or conditions, is a convenient off-the-shelf product, is able to retain its biological activity over time, is able to provide a consistent effect, including over uneven wound and body surfaces, and does not present a risk of infection.

### GENERAL DESCRIPTION OF THE INVENTION

According to the first aspect of this invention, there is provided a composition comprising amnion homogenate in admixture with a biologically acceptable excipient.

The composition provides a convenient off-the-shelf product comprising the beneficial molecular constituents of amnion, and in particular provides a composition that is useful in a wide range of applications. The composition provides an effective and consistent treatment for wounds, including slow healing wounds such as diabetic ulcers, an effective means of reducing the appearance of scars, including preventing the formation of keloid scars, for medical or cosmetic purposes, and an effective cosmetic treatment, in particular for improving the appearance of the skin or for treating one or more skin conditions. The observation that amnion homogenate improves the appearance of the skin and appears to increase fibroblast activity and collagen production (see Example 4), makes this a promising composition for a wide range of cosmetic and skin treatment purposes. In particular, the composition may be for treating dry skin, treating erythema, treating acne, including reducing acne-associated inflammation, treating sunburn or acute sun exposure, improving the smoothness of the skin, improving the appearance of skin blemishes, improving the appearance of skin aging, improving the appearance of striae gravidorum (stretch marks), improving the appearance of cellulite and improving the appearance of acute or chronic sun damage. The composition may also be applied to the body, including to a wound, for significantly longer than was previously possible. The composition also has numerous other applications such as in tissue or organ replacement, tissue engineering or as a growth medium for cell culture and especially stem cell culture.

The amnion homogenate may be formed by homogenising amnion-containing donor tissue, such as placental donor tissue. In particular, human placental donor tissue may be readily obtained from consenting donors following elective Caesarean section. Prior to donation, the donor may be screened for infections that may be present in the placental tissue, such as HIV, hepatitis B or hepatitis C. Placental donor tissue may also be obtained from non-human donors, which may include any suitable mammal such as horses, cows, pigs and sheep.

The donor tissue may be amnion and in particular may be free or substantially free of tissues other than amnion, such as the blood and other placental components such as chorion. In particular, the donor tissue may be at least 90%, at least 95%, at least 99% or at least 99.9% amnion by weight.

The amnion homogenate may comprise homogenised donor tissue along with a liquid medium. The liquid medium may be any suitable liquid, but in particular is preferably an aqueous buffer solution such as phosphate buffer saline (PBS), and may contain additives such as preservatives or protease inhibitors. The homogenised donor tissue may be present in the amnion homogenate at a concentration of at least 1% (w/v), at least 3% (w/v), at least 5% (w/v) or at least 7% (w/v), and no more than 30% (w/v), no more than 25% (w/v), no more than 20% (w/v) or no more than 15% (w/v), and in particular about 10% (w/v).

Homogenisation of the donor tissue involves breaking up the structure of the amnion in order to release the beneficial molecular constituents. Homogenisation may involve lysis of some or all of the cells of the donor tissue and breaking up the extracellular matrix. In particular, the amnion homogenate may be free or substantially free of fragments of donor tissue having a maximum dimension of greater than 50 μm, greater than 40 μm, greater than 30 μm, greater than 20 μm, greater than 10 μm or greater than 5 μm.

In addition, the amnion homogenate may be free or substantially free of debris such as cells and cell or extracellular matrix fragments. In particular, the amnion homogenate may contain no more than 10%, no more than 5%, no more than 3% or no more than 1% by weight of the cells and cell or extracellular matrix fragments present in the donor tissue.

Debris, including cells and cell or extracellular matrix fragments, may be separated from the amnion homogenate by any suitable means, such as filtration or centrifugation. In particular, the amnion homogenate may be centrifuged one or more times at relatively low speed, such as at least 400 g, at least 500 g or at least 600 g, for between 5 and 15 minutes, or about 10 minutes, in order to remove larger debris.

The amnion homogenate may also be centrifuged one or more times at relatively high speed, such as at least 6,000 g, at least 8,000 g or at least 10,000 g for between 5 and 15 minutes, or about 10 minutes, in order to remove cells and fragments of cells and extracellular matrix. This may be repeated at least twice, at least three times or at least five times.

The amnion homogenate may be sterile, and in particular may be free of infective agents such as bacteria, viruses and prions. Sterilisation processes such as heat sterilisation and gamma-irradiation may be less preferred for sterilising the amnion homogenate due to the damage that these processes may cause to the beneficial molecular constituents. However, the amnion homogenate may be sterilised by sterile filtration without causing significant damage to the beneficial molecular constituents. In particular, the amnion homogenate may be microfiltered through a filter having a pore size of no more than 2.5 μm, no more than 2.0 μm, no more than 1.5 μm or no more than 1.0 μm in order to remove microorganisms from the amnion homogenate. The amnion homogenate may further be nanofiltered through a filter having a pore size of no more than 50 nm, no more than 40 nm, no more than 30 nm or no more than 20 nm, in order to remove virus particles from the amnion homogenate.

The removal of all or substantially all debris, such as cells and cell or extracellular matrix fragments, prior to sterile filtration permits efficient sterilisation of the amnion homogenate by this method.

The amnion may also be tested for the presence of prion proteins, such as PrPs. This may be performed by any suitable method such as ELISA or Western Blot as described in the literature (Yunoki M, Tanaka H, Urayama T, Hattori S, Ohtani M, Ohkubo Y, Kawabata Y, Miyatake Y, Nanjo A, Iwao E, Morita M, Wilson E, MacLean C, Ikuta K. Prion removal by nanofiltration under different experimental conditions. Biologicals 2008; 36:27-36). Any amnion homogenate in which the presence of prion protein is detected may be discarded. Amnion from mammals such as cattle could be harvested from prion-free or CJD-free donors.

The amnion homogenate may contain one or more active beneficial molecular constituents of the amnion. In particular, the beneficial molecular constituents of the amnion include epidermal growth factor (EGF), basic fibroblast growth factor (bFGF), keratinocyte growth factor (KGF), transforming growth factor α (TGF-α), transforming growth factor β (TGF-β), nerve growth factor (NGF), hepatocyte growth factor (HGF), collagen (such as type I and type III collagen), fibronectin, nidogen and proteoglycans.

The amnion homogenate preferably comprises epidermal growth factor (EGF) at a concentration of at least 0.5 ng/ml, at least 1.0 ng/ml, at least 1.5 ng/ml or at least 2.0 ng/ml. In particular, the concentration of EGF in the amnion homogenate may range from 0.5 ng/ml to 2.0 ng/ml, or from 1.0 ng/ml to 1.5 ng/ml.

The amnion homogenate preferably comprises basic fibroblast growth factor (bFGF) at a concentration of at least 0.5 ng/ml, at least 2.5 ng/ml, at least 5 ng/ml or at least 10 ng/ml. In particular, the concentration of bFGF in the amnion homogenate may range from 0.5 ng/ml to 10 ng/ml or from 2.5 ng/ml to 5 ng/ml.

The amnion homogenate preferably comprises keratinocyte growth factor (KGF) at a concentration of at least 5 ng/ml, at least 10 ng/ml, at least 15 ng/ml or at least 20 ng/ml. In particular, the concentration of KGF in the amnion homogenate may range from 5 ng/ml to 20 ng/ml or from 10 ng/ml to 15 ng/ml.

The amnion homogenate preferably comprises transforming growth factor α (TGF-α) at a concentration of at least 0.2 ng/ml, at least 0.5 ng/ml, at least 1.0 ng/ml or at least 1.5 ng/ml. In particular, the concentration of TGF-α in the amnion homogenate may range from 0.2 ng/ml to 1.5 ng/ml or from 0.5 ng/ml to 1.0 ng/ml.

The amnion homogenate preferably comprises transforming growth factor β (TGF-β) at a concentration of at least 20 ng/ml, at least 30 ng/ml, at least 40 ng/ml or at least 50 ng/ml. In particular, the concentration of TGF-β in the amnion homogenate may range from 20 ng/ml to 50 ng/ml or 30 ng/ml to 40 ng/ml.

The amnion homogenate preferably comprises nerve growth factor (NGF) at a concentration of at least 0.1 ng/ml, at least 0.3 ng/ml or at least 0.5 ng/ml. In particular, the concentration of NGF in the amnion homogenate may range from 0.1 ng/ml to 0.5 ng/ml or from 0.3 ng/ml to 0.5 ng/ml.

The amnion homogenate preferably comprises hepatocyte growth factor (HGF) at a concentration of at least 0.5 ng/ml, at least 1.0 ng/ml or at least 1.5 ng/ml. In particular, the concentration of HGF in the amnion homogenate may range from 0.5 ng/ml to 1.5 ng/ml or from 1.0 ng/ml to 1.5 ng/ml.

The composition may comprise amnion homogenate at a concentration of at least 1% (w/w), at least 3% (w/w), at least 5% (w/w) or at least 7% (w/w), and no more than 30% (w/w), no more than 25% (w/w), no more than 20% (w/w) or no more than 15% (w/w), and in particular about 10% (w/w). The biologically acceptable excipient may make up all or substantially all of the remainder of the composition.

The biologically acceptable excipient may be any suitable material but is preferably non-toxic and non-immunogenic to avoid an adverse reaction when placed in contact with the human body.

The properties of the excipient depends on the intended use of the composition. The composition may be for topical administration, in which case the excipient may have rheological and adhesive properties that facilitate its application to and retention on the surface of the body. The excipient may further possess sufficient moisture content to enable the composition to be readily removed from the surface of the body, including from dry wounds, to enable replacement of the composition. The composition may further provide a cooling effect when applied to a surface of the body, such as by the excipient comprising volatile component. The excipient may provide a structure that allows mobilisation of different cell types to facilitate the healing effect of the composition. The composition may also be for injection or subdermal administration, in which case the excipient may have appropriate rheological and biosorption properties for this route of administration, and in particular may have a lower viscosity than in compositions for topical administration.

The composition may be a therapeutic composition. In particular, the composition may be a wound dressing, such as for dressing surgical incisions, burns, diabetic ulcers or pressure sores, or a scar dressing, for example for reducing the appearance of scars or preventing the formation of keloid scars.

The composition may also be a cosmetic composition, such as for improving the appearance of the skin or for treating the skin. In particular, the composition may be for treating dry skin, treating erythema, treating acne, including reducing acne-associated inflammation, treating sunburn or acute sun exposure, improving the smoothness of the skin, improving the appearance of skin blemishes, improving the appearance of skin aging, improving the appearance of striae gravidorum (stretch marks), improving the appearance of cellulite and improving the appearance of acute or chronic sun damage. The observation that amnion homogenate improves the appearance of the skin and appears to increase fibroblast activity and collagen production (see Example 4), makes the composition a promising composition for a wide range of cosmetic and skin treatment purposes.

Compositions for application to the body for any purpose may be in the form of a hydrogel or ointment for ease of application.

The composition may also be useful in tissue engineering, which may be performed in vivo, or in vitro, for example with the use of a bioreactor. The composition may also be for used in cell therapy, in which case the composition may further comprise viable cells, such as pluripotent stem cells. The composition may further be useful as a cell culture medium, and particularly a stem cell culture medium.

The biologically acceptable excipient may be an artificial or natural vehicle. In particular, the excipient may be an artificial or natural vehicle for delivery of the amnion homogenate. The vehicle may comprise natural or artificial bioactive peptides and bioactive components.

The biologically acceptable excipient may be a polymeric material and in particular may be a hydrophilic polymeric material. The excipient may be a hydrophilic polymer network and in particular may be a gel, including a polysaccharide based or a polypeptide based gel. In particular, the excipient may be a hydrogel, such that the compositions is in the form of a hydrogel. Suitable hydrogels include those of the type commonly used in wound treatment. The biologically acceptable excipient being a hydrogel is of particular utility not only with compositions for wound and scar treatment, but also for compositions for cosmetic purposes.

The excipient may comprise one or more water soluble polymeric materials, such as collagen, gelatin, alginate, chitosan, dextran and glycosaminoglycan, and one or more water insoluble polymeric materials, such as cotton, rayon, polyester and cellulose. In particular, the excipient may be a crosslinked hydrogel comprising one of more polymeric materials. The crosslinked hydrogel may be an alginate-gelatin hydrogel, which may be formed by reacting roughly equal quantitied or alginate dialdehyde and gelatin in the presence of borax.

The excipient may further comprise a filler, which may be present in an amount effective to improve the appearance of the composition when applied to the body. Accordingly, fillers are particularly preferred for compositions for cosmetic purposes. The filler may present in a concentration of at least 0.25% w/w, at least 0.50% w/w, at least 0.75% w/w or at least 1.00% w/w, and no greater than 5.0% w/w, 3.0% w/w or 1.5% w/w. The currently preferred filler is hyaluronic acid.

The excipient may comprise components that facilitate motility or migration of repair cells and re-construction of repairing tissue. This may allow enhanced tissue growth and re-epithelialisation, cell adhesion and collagenase IV activity. Such components may include peptides such as laminin-derived SIKVAV peptides (Ser-Ile-Lys-Val-Ala-Val) as described in the literature (Chen X, Zhang M, Wang X, Chen Y, Yan Y, Zhang L, Zhang L. Peptide-modified chitosan hydrogels promote skin wound healing by enhancing wound angiogenesis and inhibiting inflammation. Am J Transl Res 2017; 9:2352-2362).

In addition, other peptides such as natural or artificial bioactive peptides and bioactive components may be incorporated into the excipient. Such peptides may be dissolved in water or PBS using standard protocols (Seow W-Y, Salgado G, Birgitte Lane E, Hauser C A E. Transparent crosslinked ultrashort peptide hydrogel dressing with high shape-fidelity accelerates healing of full-thickness excision wounds. Sci Rep 2016; 6:32670).

The composition may comprise an oil-based excipient and may further comprise one or more surfactants or emulsifiers to facilitate admixture of the excipient with the amnion homogenate.

The composition may have a viscosity at room temperature of at least about 1,000 centipoise, at least about 5,000 centipoise, at least about 10,000 centipoise, at least about 25,000 centipoise, at least about 50,000 centipoise, at least about 100,000 centipoise or at least about 250,000 centipoise. In particular, the composition may have a viscosity of at least about 10,000 centipoise when for topical application to the body in order to facilitate its application to and retention on the body.

In addition, the composition may be non-flowable such that the composition forms a three-dimensional construct, in which case the excipient may be a cross-linked gel.

The composition may further comprise one or more additives for facilitating the preparation of the composition, enhancing the physical or mechanical properties of the composition, enhance the biological activity of the composition or providing antimicrobial properties. In particular, the composition may further comprise effective amounts of one or more of bioactive components such as growth factors or cytokines, viscosity modifiers such as carbohydrates and alcohols, surfactants, antioxidants, humectants, wetting agents, lubricants, thickeners, diluents, free-radical scavengers) plasticisers or stabilisers.

The composition may have a pH that is within the typical range of physiological pH (ie between pH 7.3 and pH 7.4) in order maintain the biological activity of the beneficial molecular constituents.

The composition may be transparent or substantially transparent in order to enable the surface to which it is applied to be directly viewed.

The composition may be combined with a scaffold in order to produce a device, such as a composite for application to the body, such as a cosmetic patch, skin treatment patch or wound dressing, or an implant, such as a tissue engineering scaffold. The tissue engineering scaffold may be a primordium for a replacement organ or tissue, such as replacement joint tissue.

A composite for application to the body may comprise a scaffold in the form of base layer to which the composition is applied. The base layer may comprise a sheet or membrane and may be substantially impermeable to the composition. The base layer may also comprise a porous material into which the composition is infused.

An implant may comprise a scaffold in the form of a bag or sachet formed of a porous material through which the beneficial molecular constituents of the composition are able to pass. An implant may comprise a porous three-dimensional scaffold into which the composition may be infused. The scaffold may include one or more additives, such as growth factors, cytokines, haemostats, platelets, preservatives or antimicrobial agents. The tissue engineering scaffold may be a primordium for a replacement organ or tissue, such as replacement joint tissue.

According to the second aspect of this invention, there is provided a method of manufacturing a composition comprising the steps of:
providing a quantity of placental donor tissue comprising amnion;
homogenising the donor tissue in order to produce an amnion homogenate; and,
admixing the amnion homogenate with a biologically acceptable excipient.

This method may be a method of manufacturing a composition according to the first aspect of this invention.

The placental donor tissue may be human placental donor tissue obtained following elective Caesarean section, or non-human placental donor tissue obtained from suitable mammals such as horses, cows, pigs and sheep.

The donor tissue may be free or substantially free of tissues other than amnion, such as the blood and other placental components such as chorion. In particular, the donor tissue may be at least 90%, at least 95%, at least 99% or at least 99.9% amnion by weight. The donor tissue is preferably washed prior to homogenisation, such as by soaking it in sterile water or sterile buffer solution such as PBS, in order to remove excess residue and blood.

The donor tissue may be combined with a liquid medium such as buffer solution, and in particular sterile PBS, during or prior to its homogenisation. The resulting amnion homogenate may comprise homogenised donor tissue at a concentration of at least 1% (w/v), at least 3% (w/v), at least 5% (w/v) or at least 7% (w/v), and no more than 30% (w/v), no more than 25% (w/v), no more than 20% (w/v) or no more than 15% (w/v), and in particular about 10% (w/v).

Homogenisation breaks up the structure of the amnion in order to release its beneficial molecular constituents and may be performed by any suitable means. The donor tissue may be cut into smaller pieces prior to homogenisation. Homogenisation may be performed with a standard laboratory homogeniser at about 5,000 to 10,000 rpm for about 10 to 15 minutes.

The method may further comprise a step of removing relatively large debris from the amnion homogenate. This may be performed by any suitable method, such as filtration or centrifugation, but is preferably performed by centrifuging the amnion homogenate one or more times at relatively low speed, such as at least 400 g, at least 500 g or at least 600 g, for between 5 and 15 minutes, or about 10 minutes, and discarding the pellet.

The method may further comprise a step of removing cells and cell or extracellular matrix fragments from the amnion homogenate. This may also be performed by filtration or centrifugation, but is preferably performed by centrifuging the amnion homogenate one or more times at relatively high speed, such as at least 6,000 g, at least 8,000 g or at least 10,000 g for between 5 and 15 minutes, or about 10 minutes, in order to remove cells and cell or extracellular matrix fragments. This may be repeated at least twice, at least three times or at least five times.

The method may further comprise a step of sterilising the amnion homogenate or the composition. Sterilisation processes such as heat sterilisation and gamma irradiation may be unsuitable for sterilising the amnion homogenate due to the damage that these processes may cause to the beneficial molecular constituents. However, the amnion homogenate may be sterilised by sterile filtration without causing significant damage to the beneficial molecular constituents. In particular, the amnion homogenate may be microfiltered through a filter having a pore size of no more than 2.5 μm, no more than 2.0 μm, no more than 1.5 μm or no more than 1.0 μm in order to remove microorganisms from the amnion homogenate. The amnion homogenate may further be nanofiltered through a filter having a pore size of no more than 50 nm, no more than 40 nm, no more than 30 nm or no more than 20 nm, in order to remove virus particles from the amnion homogenate.

The removal of all or substantially all debris, such as cells and cell or extracellular matrix fragments permits efficient sterilisation of the amnion homogenate by sterile filtration.

The amnion homogenate may also be tested for the presence of prion proteins, such as PrP^{Sc}, before and/or after sterilisation. This may be performed by any suitable method such as ELISA or Western Blot as described in the literature (Yunoki M, Tanaka H, Urayama T, Hattori S, Ohtani M, Ohkubo Y, Kawabata Y, Miyatake Y, Nanjo A, Iwao E, Morita M, Wilson E, MacLean C, Ikuta K. Prion removal by nanofiltration under different experimental conditions. Biologicals 2008; 36:27-36). Any amnion homogenate in which the presence of prion protein is detected may be discarded.

The amnion homogenate may be tested for the presence of one or more beneficial molecular constituents before it is admixed with the biologically acceptable excipient. In particular, this may involve quantifying the one or more beneficial molecular constituents, such as epidermal growth factor (EGF), basic fibroblast growth factor (bFGF), keratinocyte growth factor (KGF), transforming growth factor α (TGF-α), transforming growth factor β (TGF-β), nerve growth factor (NGF) and hepatocyte growth factor (HGF), collagen (such as type I and type III collagen), fibronectin, nidogen and proteoglycans.

The composition may comprise the amnion homogenate at a concentration of at least 1% (w/w), at least 3% (w/w), at least 5% (w/w) or at least 7% (w/w), and no more than 30% (w/w), no more than 25% (w/w), no more than 20% (w/w) or no more than 15% (w/w), and in particular about 10% (w/w) following admixing with the biologically acceptable excipient.

The biologically acceptable excipient may be a polymeric material and in particular may be a hydrophilic polymeric material. The excipient may be a hydrophilic polymer network and in particular may be a gel, such as a polysaccharide based or a polypeptide based gel. In particular, the excipient may be a hydrogel of the type commonly used in wound treatment.

The excipient may comprise one or more water soluble polymeric materials, such as collagen, gelatin, alginate, chitosan, dextran and glycosaminoglycan, and one or more water insoluble polymeric materials, such as cotton, rayon, polyester and cellulose. In particular, the excipient may be a crosslinked hydrogel comprising one of more polymeric materials. The crosslinked hydrogel may be an alginate-gelatin hydrogel, which may be formed by reacting roughly equal quantitied or alginate dialdehyde and gelatin in the presence of borax.

The method may further comprise a step of applying the composition to, or introducing the composition into, a scaffold in order to produce a device. The device may be a composite for application to the body, such as a cosmetic patch, skin treatment patch or wound dressing, or an implant, such as a tissue engineering scaffold. The tissue engineering scaffold may be a primordium for a replacement organ or tissue, such as replacement joint tissue.

The method may further comprise the introduction of one or more additives into the composition for facilitating the preparation of the composition, enhancing the physical or mechanical properties of the composition, enhancing the biological activity of the composition or providing antimicrobial properties. In particular, the method may further comprise the inclusion of one or more of bioactive components such as growth factors or cytokines, viscosity modifiers such as carbohydrates and alcohols, surfactants, antioxidants, humectants, wetting agents, lubricants, thickeners, diluents, free-radical scavengers, plasticisers or stabilisers into the composition. In addition, the method may further comprise the inclusion of one or more fillers into the composition. The one or more fillers may be present in a concentration of at least 0.25% w/w, at least 0.50% w/w, at least 0.75% w/w or at least 1.00% w/w, and no greater than 5.0% w/w, 3.0% w/w or 1.5% w/w and may include hyaluronic acid.

According to a third aspect of this invention, there is provided a kit for formation of a composition, wherein the kit comprises an amnion homogenate and, separately, a biologically acceptable excipient.

The kit may be for formation of a composition according to the first aspect of this invention.

According to a fourth aspect of this invention, there is provided an agent comprising amnion homogenate, wherein the agent is packaged with instructions to admix the agent with a biologically acceptable excipient in order to produce a composition.

The composition produced by admixing the agent with a biologically acceptable excipient may be a composition according to the first aspect of this invention.

According to a fifth aspect of this invention, there is provided a method of treating a wound, wherein the method comprises a step of topically applying to the wound a composition according to the first aspect of this invention.

The wound may be a surgical incision, burn, diabetic ulcer or pressure sore. The method may be performed on a human or a non-human animal.

The method may comprise an initial step of surgically excising a pre-existing a keloid scar, which is followed by topically applying the composition to the surgical incision thus formed.

The composition may remain applied to the wound for at least 3 days, at least 5 days or at least 7 days.

The composition is effective at treating a wound or improving the appearance of the resulting scar up to six months following injury. Accordingly, the method may comprise applying the composition to the wound up to one month, three months or six months after the wound occurred.

According to a sixth aspect of this invention, there is provided a composition according to the first aspect of this invention for use in the treatment of a wound.

The method of treatment may be a method according to the fifth aspect of this invention.

According to a seventh aspect of this invention, there is provided a composition according to the first aspect of this invention for use in the manufacture of a medicament for the treatment of a wound.

The treatment may be a method of treatment according to the fifth aspect of this invention.

According to an eighth aspect of this invention, there is provided a method of reducing scar formation from a wound, the method comprising the step of topically applying amnion to the wound.

The amnion may be in any suitable form and in particular may be unprocessed placental tissue comprising amnion or a composition comprising amnion, such as a composition according to the first aspect of this invention.

The method may be a method of reducing the appearance of cosmetic scarring, or preventing or reducing the formation of keloid scarring. The method may comprise an initial step of surgically excising a pre-existing a keloid scar, which is followed by topically applying the amnion to the surgical incision thus formed.

The amnion or composition comprising amnion homogenate may remain applied to the wound for at least 3 days, at least 5 days or at least 7 days.

Amnion is effective at improving the appearance of a scar up to six months following injury. Accordingly, the method may comprise applying the composition to the wound up to one month, three months or six months after the injury has occurred.

According to a ninth aspect of this invention, there is provided a method of improving the appearance of skin or treating a skin condition, the method comprising the step of topically applying amnion to the skin.

The amnion may be in any suitable form and in particular may be unprocessed placental tissue comprising amnion or a composition comprising amnion, such as a composition according to the first aspect of this invention.

The observation that amnion homogenate improves the appearance of the skin and appears to increase fibroblast activity and collagen production (see Example 4), makes the composition a promising composition for a wide range of cosmetic and skin treatment purposes. In particular, the method according to this aspect of the invention may be a method of treating dry skin, treating erythema, treating acne, including reducing acne-associated inflammation, treating sunburn or acute sun exposure, improving the smoothness of the skin, improving the appearance of skin blemishes, improving the appearance of skin aging, improving the appearance of striae gravidorum (stretch marks), improving the appearance of cellulite and improving the appearance of acute or chronic sun damage.

The amnion or composition comprising amnion homogenate may remain applied to the skin for at least 3 days, at least 5 days or at least 7 days.

### BRIEF DESCRIPTION OF THE DRAWINGS

FIGS. 1A-D depict the separation of amnion from chorion in donor placental tissue during term-elective Caesarean section surgery;

FIG. 2 depicts the separated amnion being cut to an appropriate size for application to a Caesarean incision;

FIG. 3 depicts a closed Caesarean incision to which amnion has been applied;

FIG. 4 depicts a composite wound dressing applied to a closed Caesarean incision comprising amnion in direct contact with the incision;

FIGS. 5-7 depict Caesarean scars in which FIGS. 5A, 6A and 7A depict scars from prior Caesarean surgery in three individuals, each of which exhibit keloid scarring, and FIGS. 5B, 6B and 7B depict Caesarean scars following the procedure set out in Example 1 in the same three individuals, none of which exhibit keloid scarring.

### EXAMPLES

The invention is described further below by way of example only, with reference to the accompanying drawings.

### Example 1—Topical Application of Amnion Reduces Cosmetic and Keloid Scarring at Caesarean Section

Amnion retrieved under sterile conditions at the time of term-elective Caesarean section was applied to the surgical incision post-surgery in order to assess its ability to reduce cosmetic and keloid scarring.

The routine procedure for closing the surgical incision was followed using a 2.0 interrupted vicryl suture to close the fat layer and a 2.0 subcuticular prolene suture to close the skin. In the case of a previous Caesarean section, the old scar was excised completely.

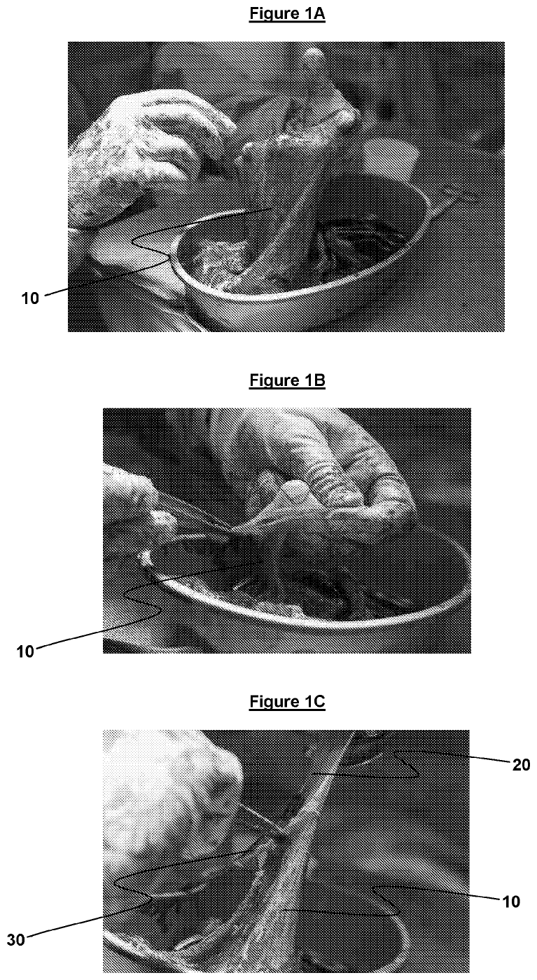

With reference to FIGS. 1A-D, at the time of Caesarean section, the donor placental tissue (10) was retained and placed on a sterile operating table and the amnion (20) was separated from the chorion (30) by hand.

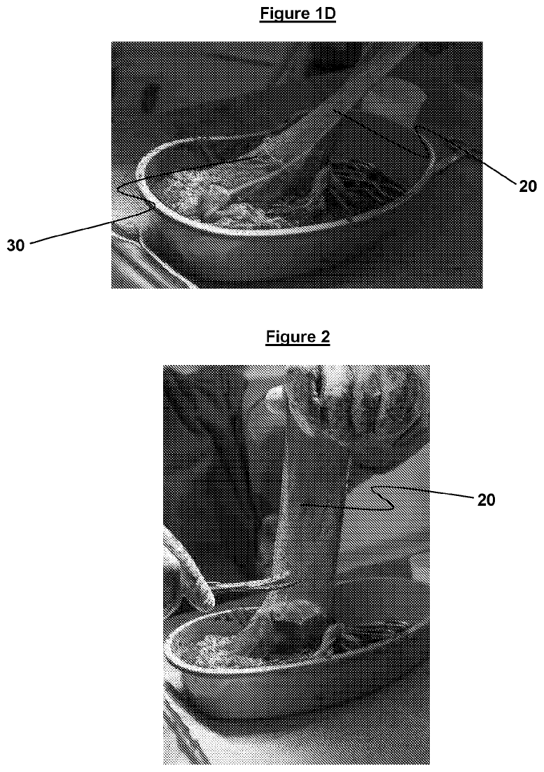

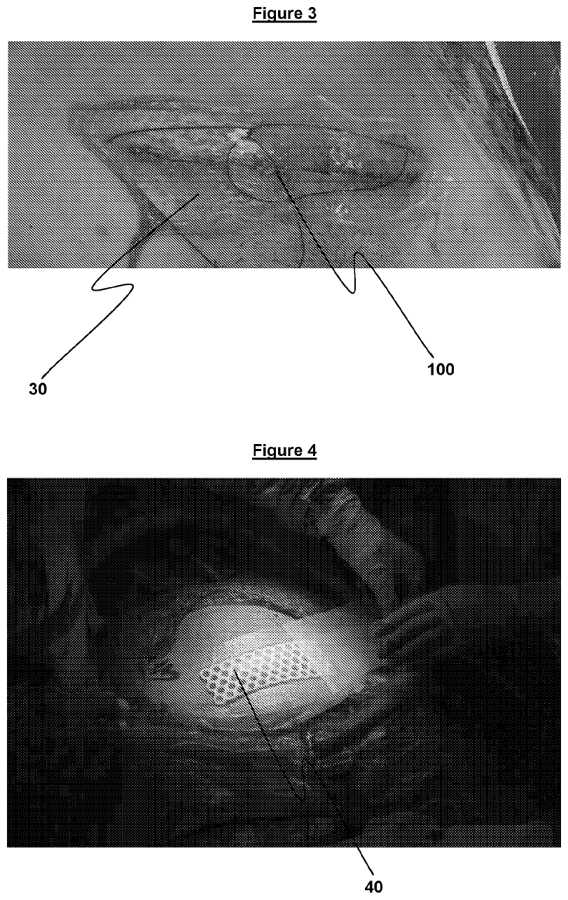

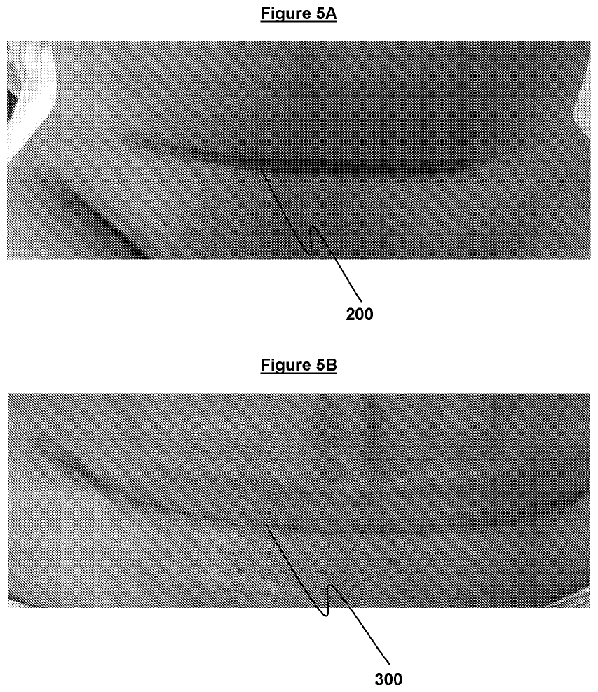

With reference to FIGS. 2-4, the amnion (20) was cut to an appropriate size for application to the Caesarean incision (100) and washed in sterile water to remove blood and other contaminants. The amnion (20) was then applied directly to the Caesarean incision (100) and covered with a honeycomb dressing (40). The resulting composite dressing remained in place for 5 days and the subcuticular stitch was then removed at day 6.

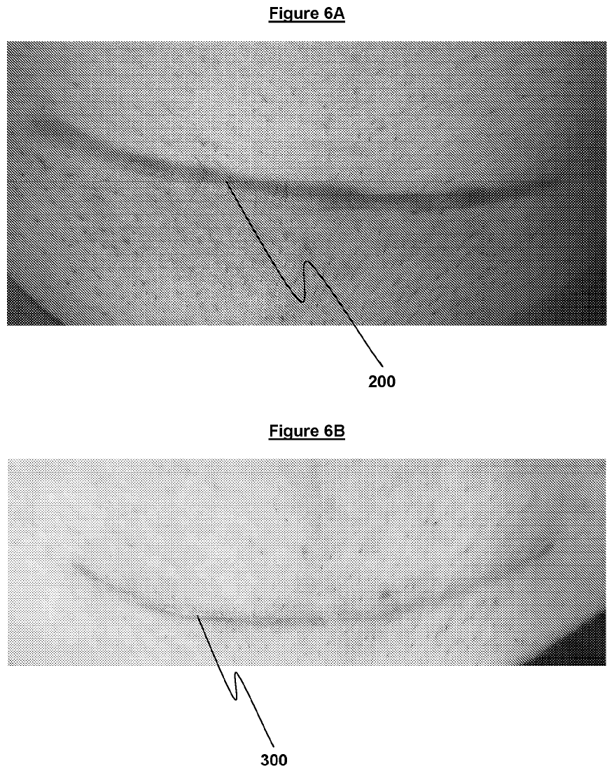

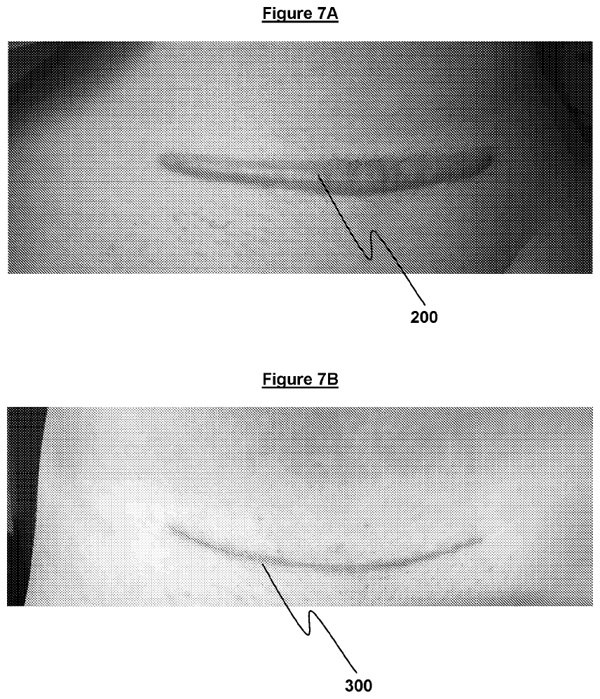

This procedure was followed for more than 50 patients, three of which already had keloid scarring from previous Caesarean surgery (see FIGS. 5-7). At 6 weeks post-surgery, photographs were taken using a Panasonic camera with a ring flash in microscopic mode.

Visual analogue scales (VAS) were completed by the patients and two independent and blinded clinicians, the results of which demonstrate a significant improvement in scar appearance.

Referring now to FIGS. 5-7, the three patients with previous keloid scarring had their previous scar (200) excised (see FIGS. 5A, 6A and 7A), and in all three cases the scar that formed following the procedure outlined above (300) did not exhibit any recurrence of keloid scarring (see FIGS. 5B, 6B and 7B).

These results demonstrate the ability of amnion to reduce cosmetic scarring and prevent the formation or recurrence of keloid scarring.

### Example 2—Preparation of Amnion Homogenate for Incorporation into a Hydrogel Wound Dressing

An amnion homogenate for incorporation into a hydrogel-based dressing was prepared from fresh placenta obtained under sterile conditions. The amnion was separated from the placenta and washed with phosphate buffered saline (PBS) to remove excess residue and blood. The amnion was then homogenised in PBS to provide an amnion homogenate at a final concentration of 10% (w/v).

The homogenate was then subjected to a low-speed centrifugation to remove larger debris, followed by further centrifugations at up to 8000 g for 10 min at 4° C., in order to remove cell debris from the homogenate, with the supernatant being recovered and the pellet discarded each time.

The supernatant was then sonicated twice for 5 min each time and the clarified and sonicated supernatant was then filtered through a Millex syringe (0.1 μm) PVDF filter (Millipore Corporation).

Western blot analysis for prion protein (PrP^{Sc}) was carried out both pre- and post-filtration as described in the literature (Yunoki M, Tanaka H, Urayama T, Hattori S, Ohtani M, Ohkubo Y, Kawabata Y, Miyatake Y, Nanjo A, Iwao E, Morita M, Wilson E, MacLean C, Ikuta K. Prion removal by nanofiltration under different experimental conditions. Biologicals 2008; 36:27-36).

### Example 3—Generation of a Hydrogel Wound Dressing Incorporating Amnion Homogenate

A novel hydrogel wound dressing was prepared incorporating the amnion homogenate prepared according to Example 2. Equal volumes of a 20% alginate dialdehyde solution in 0.1 M borax was reacted with a 15% solution of gelatin (Balakrishnan B, Mohanty M, Umashankar P R, Jayakrishnan A. Evaluation of an in situ forming hydrogel wound dressing based on oxidized alginate and gelatin. Biomaterials 2005; 26:6335-6342). Alginate dialdehyde, gelatin and borax were all used sterile. Gelation and cross-linking occurs rapidly at room temperature in order to produce a polysaccharide-based hydrogel.

Amnion homogenate was introduced into the hydrogel 10 minutes post-gelation at a ratio of between 1:1 (v/v) and 1:10 (v/v) and allowed to incorporate into the hydrogel at 37° C., in order to produce a hydrogel wound dressing incorporating amnion homogenate.

The laminin-derived SIKVAV peptide (Ser-Ile-Lys-Val-Ala-Val) chitosan hydrogel as previously reported (Chen X, Zhang M, Wang X, Chen Y, Yan Y, Zhang L, Zhang L. Peptide-modified chitosan hydrogels promote skin wound healing by enhancing wound angiogenesis and inhibiting inflammation. Am J Transl Res 2017; 9:2352-2362) may also be prepared according to the same method.

In addition, other peptides dissolved in water or PBS can be used, using standard protocols (Seow W-Y, Salgado G, Birgitte Lane E, Hauser C A E. Transparent crosslinked ultrashort peptide hydrogel dressing with high shape-fidelity accelerates healing of full-thickness excision wounds. Sci Rep 2016; 6:32670).

The hydrogel wound dressing can readily be applied topically to a wound in order to provide improved wound healing and reduced scarring by exposing the healing wound to the beneficial molecular constituents present in amnion. The hydrogel may also be combined with a scaffold or a second material or multiple materials, such as a membrane or three dimensional structure, in order to produce a composite wound dressing.

### Example 4—Wound Dressings Incorporating Amnion Homogenate Reduce Scarring and Improve the Appearance of Skin

A number of patients (n=50) undergoing Caesarean section were randomly assigned to have their Caesarean incision treated for 5 days with either a standard dressing (control group) or a wound dressings incorporating amnion homogenate (treatment group).

The patients were reviewed after 6 months and photographs of their Caesarean incisions taken using a panasonic camera with ring flash and microspcopic view. The patients were asked to score the appearance of their Caesarean scars using a visual analogue scale with a score of 100 being the best result a score of 0 being the worst result. The two clinicians were also asked to independently score the the appearance of the Caesarean scars using the same scale.

Statistical analysis was performed on the resulting scores using the Mann-Whitney non-parametric test. The mean score for the treatment group was 87.68 (range 78-97) while the mean score for the control group was 71.36 (range 62-83). A highly significant improvement was seen in the treatment group using the Mann-Whitney test (p<0.0001). These results demonstrate the efficacy of the amnion dressing in improving the appearance of the Caesarean scar following Caesarean section.

Additionally, an improvement in the appearance of the skin surrounding the Caesarean scar that was covered by the amnion dressing in the treatment group was also observed. The improvements included improved skin tone and a reduction in visible stretch marks. This appears to indicate that the amnion dressing caused fibroblast proliferation and increased collagen production in the subdermal layers of the skin.

Accordingly, wound dressings comprising amnion homogenate not only significantly improve the appearance of surgical scars but also appear to have significant cosmetic benefits for the skin, apparently by increasing fibroblast activity and collagen production. A composition incorporating amnion homogenate therefore has the potential to be beneficial in a wide range of cosmetic and skin treatment applications.

1. A composition comprising amnion homogenate in admixture with a biologically acceptable excipient.

2. The composition of claim 1, wherein the amnion homogenate is human amnion homogenate.

3. The composition of claim 1, wherein the amnion homogenate is free or substantially free of tissues other than amnion.

4. The composition of claim 1, wherein the amnion homogenate comprises homogenised amnion-containing donor tissue and a liquid medium.

5. The composition of claim 4, wherein the amnion homogenate comprises homogenised amnion-containing donor tissue at a concentration of at least 1% (w/v) and no more than 30% (w/v).

6. The composition of claim 1, wherein the composition is free or substantially free of fragments of donor tissue having a maximum dimension of greater than 50 μm.

7. The composition of claim 1, wherein the amnion homogenate is free or substantially free of cells and cell or extracellular matrix fragments.

8. The composition of claim 1, wherein the amnion homogenate comprises no more than 10% by weight of the cells and cell or extracellular matrix fragments present in the donor tissue.

9. The composition of claim 1, wherein the composition comprises at least one of epidermal growth factor (EGF), basic fibroblast growth factor (bFGF), keratinocyte growth factor (KGF), transforming growth factor α (TGF-α), transforming growth factor β (TGF-β), nerve growth factor (NGF) and hepatocyte growth factor (HGF), collagen, fibronectin, nidogen and proteoglycans.

10. The composition of claim 1, wherein the composition comprises amnion homogenate at a concentration of at least 1% (w/w), at least 3% (w/w), at least 5% (w/w) or at least 7% (w/w), and no more than 30% (w/w), no more than 25% (w/w), no more than 20% (w/w) or no more than 15% (w/w), and in particular about 10% (w/w).

11. The composition of claim 1, wherein the composition is a therapeutic composition or a cosmetic composition.

12. The composition of claim 11, wherein the composition is a wound dressing, a scar dressing, a topical hydrogel or a topical ointment.

13. The composition of claim 1, wherein the excipient is a hydrophilic polymeric material.

14. The composition of claim 13, wherein the excipient is a polysaccharide based or a polypeptide-based gel.

15. The composition of claim 13, wherein the excipient is a hydrogel.

16. The composition of claim 13, wherein the excipient comprises one or more water soluble polymeric materials and/or one or more water insoluble polymeric materials.

17. The composition of claim 1, wherein the composition has a viscosity at room temperature of at least about 1,000 centipoise.

18. The composition of claim 1, wherein the composition is in the form of a three-dimensional construct comprising a cross-linked gel.

19. The composition of claim 1, wherein the composition further comprises an effective amount of one of more of growth factors, cytokines, viscosity modifiers, surfactants, antioxidants, humectants, wetting agents, lubricants, thickeners, diluents, free-radical scavengers, plasticisers and stabilisers.

20. A device comprising the composition of claim 1 and a component scaffold to which the composition is applied or into which the composition is infused.

21. The device of claim 20, wherein the scaffold comprises an effective amount of one or more of growth factors, cytokines, haemostats, platelets, preservatives or antimicrobial agents.

22. A method of manufacturing a composition comprising the steps of:
providing a quantity of placental donor tissue that comprises amnion;
homogenising the donor tissue in order to produce an amnion homogenate; and,
admixing the amnion homogenate with a biologically acceptable excipient.

23. (canceled)

24. The method of claim 22, wherein the amnion homogenate may comprise homogenised donor tissue at a concentration of at least 1% (w/v) and no more than 30% (w/v).

25. The method of claim 22, wherein the method further comprises centrifuging the amnion homogenate one or more times at a speed of at least 400 g for between 5 and 15 minutes.

26. The method of claim 22, wherein the method further comprises centrifuging the amnion homogenate one or more times at a speed of at least 6,000 g for between 5 and 15 minutes.

27. The method of claim 22, wherein the method further comprises a sterilisation step.

28. The method of claim 27, wherein the amnion homogenate is sterile filtered through a filter having a pore size of no more than 2.5 μm.

29. The method of claim 27, wherein the amnion homogenate is sterile filtered through a filter having a pore size of no more than 50 nm.

30. A kit for formation of a composition or a device, wherein the kit comprises an amnion homogenate and, separately, a biologically acceptable excipient.

31. (canceled)

32. An agent comprising amnion homogenate, wherein the agent is packaged with instructions to admix the agent with a biologically acceptable excipient in order to produce a composition.

33. (canceled)

34. A method of treating a wound, wherein the method comprises a step of topically applying to the wound the composition of claim 1.

35. The method of claim 34, wherein the wound is a surgical incision, burn, diabetic ulcer or pressure sore.

36. The method of claim 34, wherein the method further comprises an initial step of surgically excising a pre-existing a keloid scar.

37-38. (canceled)

39. A method of reducing scar formation from a wound, the method comprising the step of topically applying amnion to the wound.

40. The method of claim 39, wherein the amnion is in the form of a composition comprising amnion homogenate in admixture with a biologically acceptable excipient.

41. The method of claim 39, wherein the method further comprises an initial step of surgically excising a pre-existing a keloid scar.

42. A method of improving the appearance of skin or treating a skin condition, the method comprising the step of topically applying amnion to the skin.

43. The method of claim 42, wherein the amnion is in the form of a composition comprising amnion homogenate in admixture with a biologically acceptable excipient.

44. The method of claim 42, which is a method of treating dry skin, treating erythema, treating acne, reducing acne-associated inflammation, treating sunburn, treating acute sun exposure, improving the smoothness of the skin, improving the appearance of skin blemishes, improving the appearance of skin aging, improving the appearance of striae gravidorum, improving the appearance of cellulite or improving the appearance of acute or chronic sun damage.
